(19) United States
(12) Patent Application Publication
(10) Pub. No.: US 2026/0294466 A1
(43) Pub. Date: Oct. 1, 2026

(54) ENDOSCOPIC SNARE DEVICE

(71) Applicant: United States Endoscopy Group, Inc., Mentor, OH (US)

(72) Inventors: Alex Uspenski, Chardon, OH (US); John P. Winstanley, Madison, OH (US); Cynthia Ranallo, Eastlake, OH (US); Christopher J. Kaye, Berea, OH (US); Reza Mohammadpour, Willoughby Hills, OH (US); Michael Simenc, Sagamore Hills, OH (US); Craig Moore, Pepper Pike, OH (US); Tony Martella, Mentor, OH (US); Nicholas R. Marciniak, Mentor, OH (US)

(73) Assignee: United States Endoscopy Group, Inc., Mentor, OH (US)

(21) Appl. No.: 19/704,664

(22) Filed: Jun. 11, 2026

Related U.S. Application Data
(63) Continuation of application No. 17/193,700, filed on Mar. 5, 2021, now abandoned.
(60) Provisional application No. 62/989,189, filed on Mar. 13, 2020.
(60) Provisional application No. 63/039,097, filed on Jun. 15, 2020.

(51) Int. Cl. A61B 17/3205; A61B 17/221

(52) U.S. Cl. CPC A61B 17/32056; A61B 17/221; A61B 2017/2212

(57) ABSTRACT

A tissue removal tool includes a body, a conduit, a loop, and a transmitting assembly. The conduit is attached to the body and defines an inner lumen, where the inner lumen has a proximal portion having a first width and a distal portion having a second width that is greater than the first width. The loop is movable between an open position in which the loop is at least partially disposed outside of the conduit and a closed position in which the loop is disposed within the conduit. The transmitting assembly includes a handle and a link. The handle is connected to the body, and the link has a first end attached to the handle and a second end attached to the loop such that movement of the handle causes the loop to move between the open and closed positions.

### CROSS-REFERENCE TO RELATED APPLICATIONS

The present application is a continuation of U.S. patent application Ser. No. 17/193,700, filed on Mar. 5, 2021, which claims benefits and priority to U.S. Provisional Patent Application No. 62/989,189, filed on Mar. 13, 2020, and U.S. Provisional Patent Application No. 63/039,097, filed on Jun. 15, 2020, the entire disclosures of which are incorporated herein by reference.

### TECHNICAL FIELD

The present subject matter relates generally to endoscopic snare devices.

### BACKGROUND

Polypectomy, or the removal of polyps, has become one of the most common endoscopic procedures in gastrointestinal endoscopy today. Its relative simplicity often belies the effectiveness of the procedure at preventing colorectal cancer. The National Polyp Study (NPS) demonstrated a reduction in the incidence of colorectal cancer of 76%-90% following colonoscopic polypectomy. Standard practice for polypectomy has been to use an electrocautery or “hot” snare to remove polyps due to the fact that it reduces the risk of bleeding as a result of the coagulation effect created by the current. Electrocautery, however, can create inadvertent damage to healthy tissue and may not be necessary for smaller sized polyps where bleeding risk is not a concern.

A “cold” snare, such as US Endoscopy Group Inc.'s Exacto® snare, is designed for polypectomy procedures where diminutive polyps are encountered. It enables a clean cut that reduces polyp “fly away” from the resection site. Studies comparing Exacto® snare to hot snares have shown that, for diminutive polyps in the 3-8 mm range, there is no difference in postpolypectomy bleeding and it requires less time to use than hot snares while being just as safe and effective.

### SUMMARY

An exemplary embodiment of a tissue removal tool includes a body, a conduit, a reinforcement member, a loop, and a transmitting assembly. The conduit is attached to the body, and the reinforcement member is configured to reinforce at least a portion of the conduit. The loop is movable between an open position in which the loop is at least partially disposed outside of the conduit and a closed position in which the loop is at least partially disposed within the conduit. The transmitting assembly includes a handle and a link. The handle is connected to the body, and the link has a first end attached to the handle and a second end attached to the loop such that movement of the handle causes the loop to move between the open and closed position.

Another exemplary embodiment of a tissue removal tool includes a body, a conduit, a loop, and a transmitting assembly. The conduit is attached to the body and defines an inner lumen, in which the inner lumen has a proximal portion having a first width and a distal portion having a second width that is greater than the first width. The loop is movable between an open position in which the loop is at least partially disposed outside of the conduit and a closed position in which the loop is at least partially disposed within the conduit. The transmitting assembly includes a handle and a link. The handle is connected to the body, and the link has a first end attached to the handle and a second end attached to the loop such that movement of the handle causes the loop to move between the open and closed position.

Another embodiment of a tissue removal tool includes a body, a conduit, a loop, and a transmitting assembly. The conduit is attached to the body and defines an inner lumen. The inner lumen includes a chamfered distal end having a width that gradually increases along a length of the chamfered distal end from a first width to a second width. The loop is movable between an open position in which the loop is at least partially disposed outside of the conduit and a closed position in which the loop is disposed within the conduit. The transmitting assembly includes a handle and a link, where the handle is connected to the body, and where the link has a first end attached to the handle and a second end attached to the loop such that movement of the handle causes the loop to move between the open and closed positions.

Another exemplary embodiment of a tissue removal tool includes a body, a conduit, a loop, a transmitting assembly, and a connector. The conduit is attached to the body. The loop is movable between an open position in which the loop is at least partially disposed outside of the conduit and a closed position in which the loop is at least partially disposed within the conduit. The transmitting assembly includes a handle and a link. The handle is connected to the body, and the link has a first end attached to the handle and a second end attached to the loop such that movement of the handle causes the loop to move between the open and closed position. The connector is for connecting the second end of the link to the loop. The connector has a first opening for receiving the link and a second opening for receiving the loop, in which a first plane extends through a center of the first opening and a second plane extends through a center of the second opening, and in which the first plane is offset from and parallel to the second plane.

An exemplary embodiment of a connector for connecting a link of a tissue removal tool to a loop of a tissue removal tool includes a body, a first opening, and a second opening. The first opening is for receiving the link, and the second opening is for receiving the loop. A first plane extends through a center of the first opening, and a second plane extends through a center of the second opening. The first plane can be offset from and perpendicular to the second plane.

### BRIEF DESCRIPTION OF THE DRAWINGS

The features and advantages of the general inventive concepts will become apparent from the following detailed description made with reference to the accompanying drawings.

FIG. 1 illustrates an exemplary embodiment of a tissue removal tool, showing a loop of the tissue removal tool in an open position;

FIG. 2 is an enlarged sectional view of a portion of the tissue removal tool of FIG. 1, showing the loop in a closed position within a conduit;

FIG. 3 illustrates an exemplary embodiment of a loop for a tissue removal tool, in which the loop has a general cable form design;

FIG. 4 illustrates another exemplary embodiment of a loop for a tissue removal tool, in which the loop has a general monofilament form design;

FIG. 5 illustrates another exemplary embodiment of a loop for a tissue removal tool, in which a distal cutting section and a proximal portion of the loop are formed from separate wires;

FIGS. 6a-6b illustrate another exemplary embodiment of a loop for a tissue removal tool, in which the loop has microtomes or cutting elements;

FIG. 7 illustrates another exemplary embodiment of a loop for a tissue removal tool, in which the loop has a torsion tip;

FIG. 7a illustrates an exemplary embodiment of a torsion tip for the loop of FIG. 7;

FIG. 7b illustrates an exemplary embodiment of a loop for a tissue removal tool, in which the loop has a coined distal tip;

FIG. 7c illustrates a side view of the loop of FIG. 7b;

FIG. 7 d illustrates a side view of the distal tip of FIG. 7 c shown in detail A of FIG. 7 c;

FIG. 8a illustrates another exemplary embodiment of a loop for a tissue removal tool, in which the loop has separate loop elements;

FIG. 8b illustrates another exemplary embodiment of a loop for a tissue removal tool, in which the loop has separate loop elements;

FIG. 8c illustrates another exemplary embodiment of a loop for a tissue removal tool, in which the loop has separate loop elements;

FIGS. 9a-9b illustrate another exemplary embodiment of a loop for a tissue removal tool, in which the loop has a tapered or ground portion;

FIG. 10 illustrates another exemplary embodiment of a loop for a tissue removal tool, in which the loop has a coined proximal portion;

FIG. 11 illustrates another exemplary embodiment of a loop for a tissue removal tool, in which the loop has a coined or non-coined proximal portion and a coined distal portion;

FIGS. 12a-12b illustrate another exemplary embodiment of a loop for a tissue removal tool, in which the loop has a plurality of coined sections;

FIG. 13a illustrates another exemplary embodiment of a loop for a tissue removal tool, in which the loop has mounted cutting elements;

FIG. 13b illustrates the mounted cutting elements for the loop of FIG. 13a;

FIG. 14 is another exemplary embodiment of a loop for a tissue removal tool;

FIG. 15 illustrates another exemplary embodiment of a tissue removal tool;

FIG. 16 illustrates another exemplary embodiment of a tissue removal tool;

FIG. 16a illustrates the tissue removal tool of FIG. 16 connected to an exemplary embodiment of an endoscopic device;

FIG. 16b illustrates the positioning of a conduit and reinforcing member of the tissue removal tool of FIG. 16 within an inlet of a channel of the endoscopic device of FIG. 16a;

FIG. 17 is a cross-sectional view of the tissue removal tool of FIG. 16, shown along the lines 17-17 shown in FIG. 16;

FIG. 18 is a cross-sectional view of the tissue removal tool of FIG. 16, shown along the lines 18-18 shown in FIG. 17;

FIG. 19 illustrates another exemplary embodiment of a tissue removal tool;

FIG. 19a illustrates the tissue removal tool of FIG. 19 connected to an exemplary embodiment of an endoscopic device;

FIG. 19b illustrates the positioning of a conduit and reinforcing member of the tissue removal tool of FIG. 19 within an inlet of a channel of the endoscopic device of FIG. 19a;

FIG. 20 is a side view of another exemplary embodiment of a tissue removal tool, in which the tissue removal tool has a connector for connecting a link to a loop;

FIG. 21 is a top view of the tissue removal tool of FIG. 20;

FIG. 22 is a perspective view of an exemplary embodiment of a connector for the tissue removal tool of FIG. 21;

FIG. 23 is a side view of the connector of FIG. 22;

FIG. 24 illustrates another exemplary embodiment of a connector for a tissue removal tool of FIG. 20;

FIG. 25 illustrates an exemplary embodiment of a conduit for a tissue removal tool;

FIG. 25A illustrates an exemplary embodiment of a chamfered distal end for the conduit of FIG. 25; and

FIG. 26 illustrates another exemplary embodiment of a conduit for a tissue removal tool.

### DETAILED DESCRIPTION OF INVENTION

This Detailed Description merely describes exemplary embodiments in accordance with the general inventive concepts and is not intended to limit the scope of the invention or the claims in any way. The invention as described by the claims is broader than and unlimited by the exemplary embodiments set forth herein, and the terms used in the claims have their full ordinary meaning.

The general inventive concepts will now be described with occasional reference to the exemplary embodiments of the invention. This general inventive concept may, however, be embodied in different forms and should not be construed as limited to the embodiments set forth herein.

Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts. As used in this detailed description and the appended claims, the singular forms “a,” “an,” and “the” are intended to include the plural forms as well, unless the context clearly indicates otherwise.

Unless otherwise indicated, all numbers such as, for example, numbers or number ranges expressing measurements or physical characteristics, used in the specification and claims are to be understood as being modified in all instances by the term “about.” Accordingly, unless otherwise indicated, the numerical properties set forth in the specification and claims are approximations that may vary depending on the suitable properties sought to be obtained in embodiments of the invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

The present application describes various components as being “proximal” or “distal.” As used herein, the term “proximal” refers to a portion of a component that is situated nearer to the center of the body of a tissue removal tool, or to a direction toward the center of the body of the tissue removal tool, unless the context clearly indicates otherwise. As used herein, the term “distal” refers to a portion of a component that is situated away from the center of the body of a tissue removal tool, or to a direction away the center of the body of the tissue removal tool, unless the context clearly indicates otherwise.

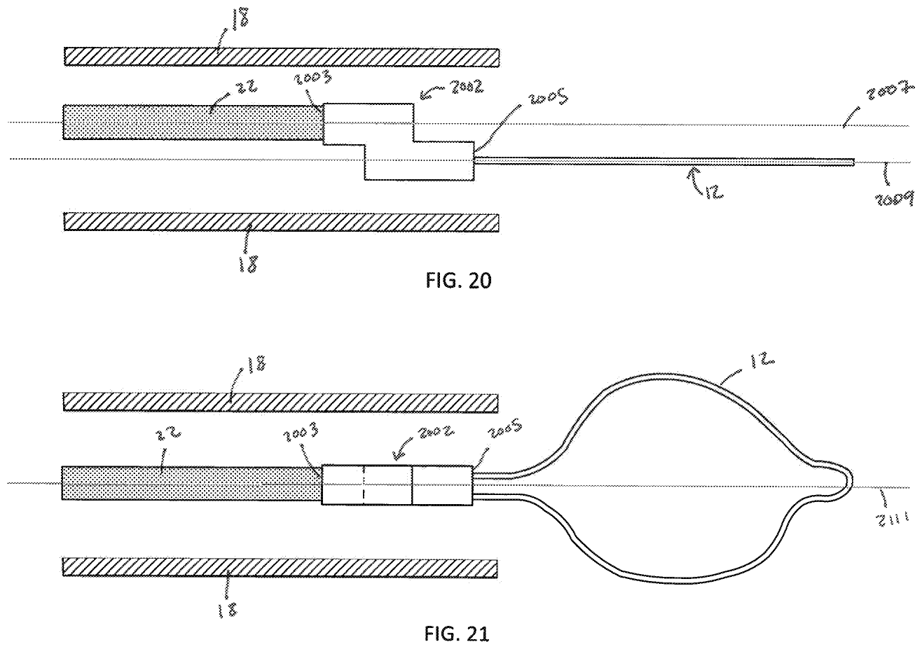

The present application describes a tissue removal tool for use with an endoscope, in which the tissue removal tool has a loop, a handle, and a link. The loop is movable between an open position and a closed position, where the loop is defined in the open position by a proximal portion and a distal portion. The loop may be constructed from a piece of wire. A first end of the link is attached to the handle and a second end of the link is attached to the loop such that the loop can be moved between the open and closed positions by action of the handle. In certain embodiments, the tissue removal tool includes a conduit for housing the link and the loop (when the loop is in the closed position). The conduit may be reinforced by a reinforcement member to prevent the conduit from buckling, bending, or kinking. In some embodiments, the link is connected to the loop by a connector that allows the loop to have a greater tendency to lay down in a desired position on a patient's tissue during use of the tool, which can increase the amount of tissue removed from a patient. That is, the connector maintains the loop in a substantially flat position (rather than an angled position), which allows the loop to engage the base of a polyp and make a substantially straight cut through the polyp, thus removing a larger portion of the polyp. The connector can take any suitable form that allows the loop to have a greater tendency to lay down in a desired position, such as, for example, any form described in the present application for connector 2002 shown in FIGS. 20-24.

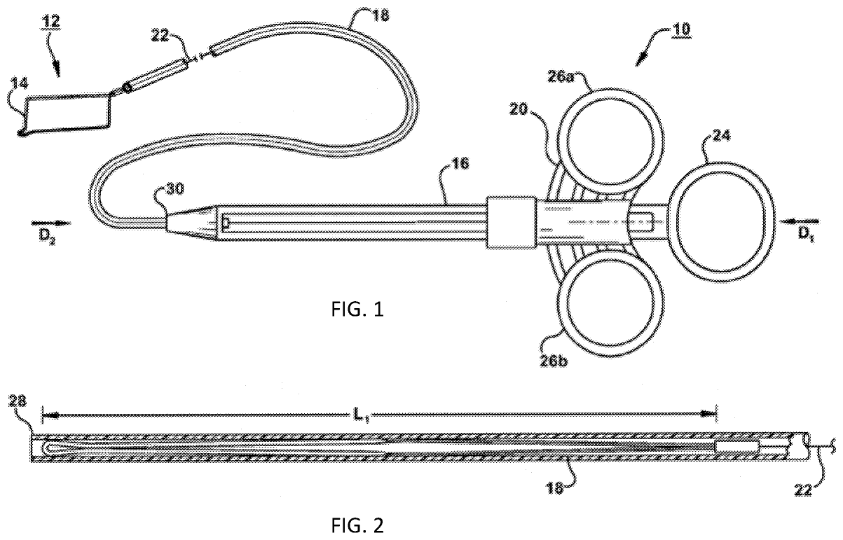

Referring to FIG. 1, an exemplary embodiment of a tissue removal tool 10 includes a loop 12 formed by a piece of wire 14. A wire of any suitable material may be used to form the loop 12, such as for example, a metal, such as stainless steel, nitinol, plastic or carbon nanotube, glass fiber, or hybrid technology. The loop 12 may also be formed by a monofilament or a cable. The loop may include one or more sizes, stages, or configurations. The variations in the shape of the wire 14 shape and orientation along the length of the wire 14 may be made by bending or twisting a preformed wire into the desired shape and orientation, or the wire 14 may be originally manufactured to have the desired shape and orientation, such as, for example, the wire 14 may be from a preformed plastic piece.

In certain embodiments, a suitable material for the wire 14 will be flexible and have memory to allow for deployment into a first state or configuration and retrieval of the loop 12 into a second state or configuration. The wire 14 may form a plurality of segments separated by collapse-resistant bends to define an opening of the loop 12. The loop 12 is movable between an open or deployed position and a closed or retrieved position. The loop 12 is illustrated in an open position in FIG. 1. In the illustrated embodiment, the loop is polygon-shaped and, specifically, generally diamond-shaped. The loop 12 may be formed with additional bend points to allow it to pen to different sizes, stages, or configurations. In FIG. 2, the loop 12 is shown in a closed position within a distal portion of the tissue removal tool 10. As discussed herein, the loop 12 is within a conduit 18 when in a closed position. In certain embodiments, the loop 12 has a length L₁ within the conduit when in the closed position.

The tool 10 may include a support assembly and a transmitting system for moving the loop 12 between the open and closed positions. In the illustrated embodiment, the support assembly includes body 16 and a conduit 18. The transmitting assembly includes a handle 20 and a link 22, in which one end of the link 22 is fixed to the handle 20 and a second end of the link 22 is remote from the body 16 such that the link 22 extends substantially through a length of the conduit 18. The conduit 18 may be any suitable, small-diameter tube formed of a low-friction flexible material such as, for example, polytetrafluorethylene, high density polyethylene, polyether block amide, or other comparable materials. A proximal end 30 of the conduit 18 is connected to the body 16, and the conduit 18 defines a lumen with an opening at a distal end 28 such that the loop can be deployed from and retrieved into the distal end 28.

In certain embodiments, the tissue removal tool 10 is a cold tissue removal tool that does not utilize electricity to heat the loop 12 during the removal of tissue from a patient. In some embodiments, the tissue removal tool is a hot tissue removal tool that utilizes electricity to heat the loop during removal of tissue from a patient. For example, the tissue removal tool 10 may be electrically connected to an electrosurgical generator such that electrical energy is transferred from the electrosurgical generator to the loop 12 to heat the loop 12. In certain embodiments, this electrical connection may include connections between the loop 12, the link 22, a hypotube, and a connector. In various embodiments, the tissue removal tool 10 may be used as either a cold tissue removal tool or a hot tissue removal tool such that the loop 12 can be heated when needed and remain cold when needed.

Referring to FIG. 1, the body 16 includes a fixed holding member 24 (e.g., a ring) such that a user can grip the body 16 during use of the tool 10. The handle 20 is movable relative to the body 16 such that a user can move the handle 20 to cause the loop to move between the open and closed positions. The loop 12 may open to several stages or widths. In certain embodiments, the loop opens to the several stages or widths from smallest to largest. The loop 12 may include two or more stages, such as three or more stages, such as four or more stages. In embodiments having three stages, the first stage may have a width of between about 5 mm and about 10 mm, such as about 6 mm; the second stage may have a width of between about 10 mm and about 15 mm, such as about 18 mm, and the third stage may have a width of between about 15 mm and about 30 mm, such as about 18 mm.

The handle 20 can include one or more holding members 26a, 26b (e.g., rings) such that a user can grip the handle 20 to move the handle relative to the body 16. A link 22 is connected to the handle 20 for transferring axial motion from the handle 20 to other parts of the device (e.g., the loop 12). The link 22 may be constructed of any suitable rigid material, and may be solid, hollow, or any suitable elongated object or combination of objects. The link 22 may be one piece or formed from a series of pieces and connections, such as for example, hypodermic tubes, swage connections, and cables. In the illustrated embodiment, the link 22 has a first end connected to the handle 20 and a second end connected to the loop 12. As shown in the drawings, the link extends substantially through the conduit 18.

In the illustrated embodiment, the handle 20 is mounted over an elongated section of the body 16 and is movable relative to the body in the direction D₁ to deploy the loop 12 out of the conduit 18 and into the open position. The handle 20 is movable in an opposing direction D₂ to close the loop 12 by moving the loop into the conduit 18. For example, an operator may place a finger in each of the holding members 26a, 26b of the handle 20 and a thumb of the same hand in the fixed holding member 24 of the body 16. By the operator moving the two fingers engaging the holding members 26a, 26b in the direction D₁, the handle 20 will cause the link 22 to move in a distal direction relative to the body 16 such that the loop 12 deploys from the conduit 18 and into the open position. By the operator moving the two fingers engaging the holding members 26a, 26b in the direction D₂, the handle 20 will cause the link to move in a proximal direction relative to the body 16 such that the loop 12 is retrieved in the conduit 18 and into the closed position.

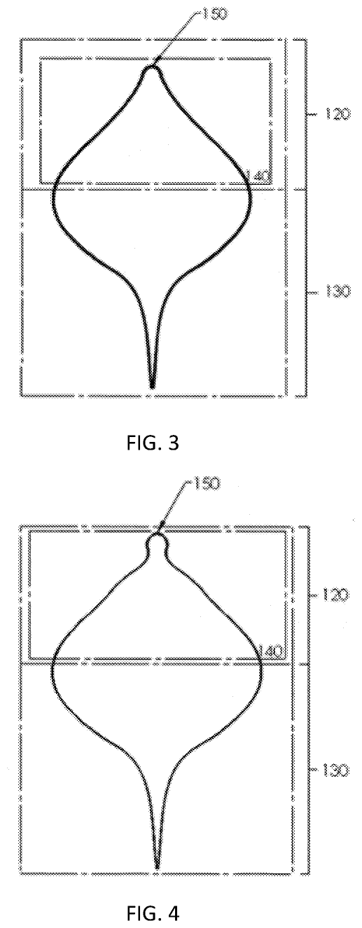

FIG. 3 shows a general form for a cable cold cutting snare loop, and FIG. 4 shows a general form for a monofilament cold cutting snare loop. The cable loop form shown in FIG. 3 dissipates stress more uniformly over itself than the monofilament loop form shown in FIG. 4 because the cable loop is formed of individual strands (not shown) that can move relative to one another. These individual strands allow the cable loop form to be flexible, which allows the loop 12 to be placed more easily during use and reduces deformation of the loop resulting from forces acting on the loop during placement and resection. Comparatively, the monofilament snare is composed of a single filament that tends to more readily deform at the bend points. This deformation can be mitigated by including a rounded tip (e.g., a semi-circular shaped tip, a curved tip, a bent tip, etc.) at the distal end 150 of the loop 12. The rounded tip is configured to efficiently handle a load because it distributes the forces evenly over a greater length of the distal end 150 of the loop 12, which reduces deformation of the loop 12 during normal use.

Still referring to FIGS. 3 and 4, the loops 12 include a distal portion 120 and a proximal portion 130. The distal portion 120 of the loops 12 may include a cutting section 140. In certain embodiments, the cutting section 140 covers roughly about ⅓ to about ½ a length of the loop 12, and may be continuous or discrete. In some embodiments, the cutting section 140 extends about 1-5 mm out from the distal end 150 in either direction. In some embodiments, the cutting section 140 extends about 5 mm to about 15 mm out from the distal end 150 in either direction. In some embodiments, the cutting section 140 extends about 10 mm out from the distal end 150 in both directions. In some embodiments, the cutting section 140 extends about 5 mm to 15 mm out from the distal end 150 in both directions. The cutting section 140 needs to be thin enough to provide cutting pressure to tissue. The cutting section 140 may have a diameter of about 0.36 mm or thinner so as to enable cold cutting of tissue. In some embodiments, the diameter is about 0.30 or thinner. The cutting mostly occurs on the distal portion 120 of the snare by virtue of tensile mechanics of the snare as it is retracted into the conduit 18. The distal tip 150 may also be optimized to dissipate stress uniformly so as not to deform during retraction.

In various embodiments, the proximal portion 130 of the loop 12 may not perform cutting, but the proximal portion 130 may be configured to provide assistance and support to encircle and recruit tissue for cutting. Increasing the stiffness of the proximal portion 130 allows for greater indentation of the tissue which improves tissue recruitment. That is, a larger stiffness of the proximal portion allows a user to apply more force onto the tissue and causing an indentation of the tissue, which increases the frictional forces keeping the tissue from being disengaged from the snare upon closing. The stiffer proximal portion 130 can be about ⅔ to about ½ a length of the loop 12. In some embodiments, the proximal portion 130 may vary in stiffness along its length.

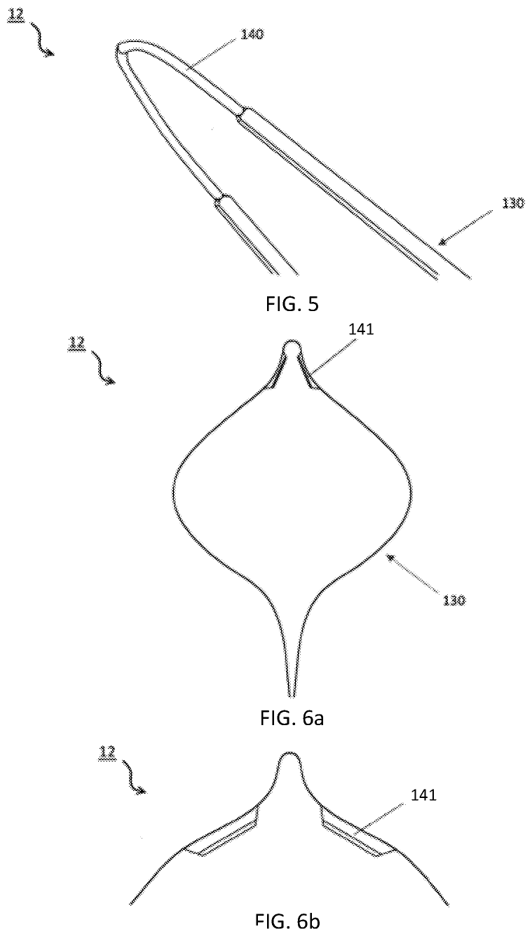

Referring to FIG. 5, in certain embodiments, the proximal portion 130 includes a flat wire or coined cable, and the cutting section 140 may include a thinner wire than the proximal portion 130. These separate portions may be joined together by welding, gluing, crimping, swaging, soldering, or other techniques familiar to those skilled in the art.

Referring to FIGS. 6a and 6b, the cutting section 140 may include cutting blades 141 that are disposed on an otherwise non-cutting snare wire.

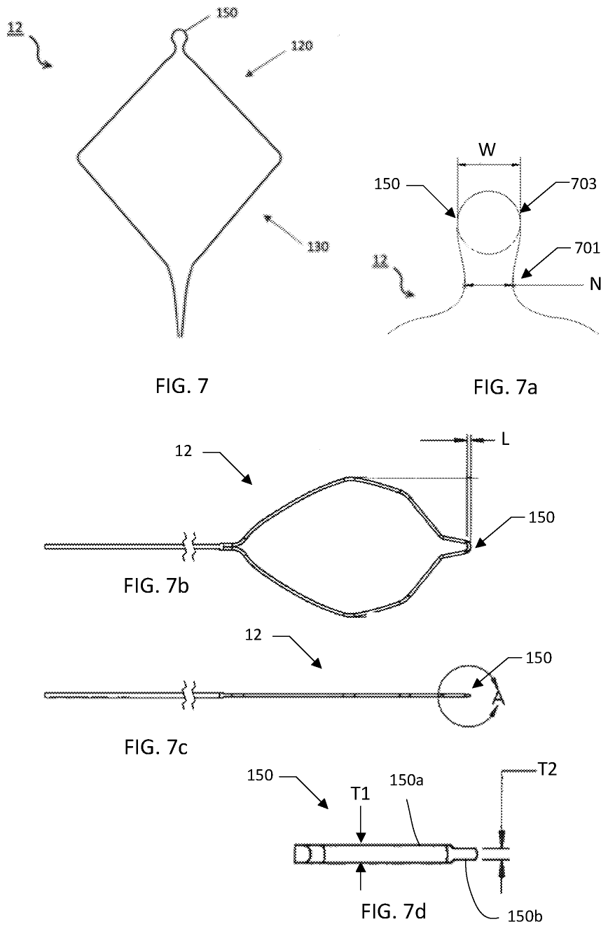

Referring to FIG. 7, in some embodiments, the loop 12 may be a solid rectangular wire monofilament loop with a torsion tip 150 to preserve its shape. While the illustrated embodiment of the monofilament loop 12 has a rectangular shape, it should be understood that a monofilament loop 12 can be any suitable shape, such as, for example, the round shape shown in FIG. 4. In certain embodiments, the torsion tip 150 may have a circular or partial-circular shape, which optimizes the bending strength of the loop 12 while also minimizing the thickness of the tip 150. For example, the tip 150 may have a shape of less than or equal to 360 degrees of a circle, such as less than or equal to 270 degrees of a circle, such as less than or equal to 180 degrees of a circle. In addition, the torsion tip 150 may have a shape of between 90 degrees of a circle and 270 degrees of a circle, such as about 180 degrees of a circle.

Referring to FIG. 7a, in some embodiments, the torsion tip 150 of a monofilament loop 12 may include a proximal portion 701 and a distal portion 703. In certain embodiments, the proximal portion 701 has a minimum width N, and the distal portion 703 has a maximum width W that is greater than the minimum width N. For example, the maximum width W may be between 5% greater and 100% greater than the minimum width N, such as between 10% and 90% greater, such as between about 25% and 75% greater, such as about 50% greater. The maximum width W may be less than or equal to 2.5 mm, such as less than or equal to 2 mm, such as less than or equal to 1.5 mm, such as less than or equal to 1 mm. The minimum width N may be less than or equal to 1.5 mm, such as less than or equal to 1 mm, such as less than or equal to 0.75 mm, such as less than or equal to 0.5 mm, such as less than or equal to 0.25 mm. The maximum width W of the distal portion 703 being greater than the minimum width N of the proximal portion 701 causes the stress to be evenly distributed over the distal end of the loop, which prevents the loop 12 from deforming. In some embodiments, the distal portion 703 may have a partial-circular shape (e.g., a semi-circular shape) and the maximum width W is equal to the diameter of the partial-circular shape.

FIGS. 7b-7d show an exemplary embodiment of a loop 12 having a coined distal tip 150. The loop 12 and distal tip 150 may include the features of any tissue removal tool 10 disclosed in the present application. In certain embodiments, the loop 12 may be a cable loop. In other embodiments, the loop 12 may be a monofilament loop. The distal tip 150 may have a proximal portion 150a that has a first thickness T1 and a distal portion 150b that has a second thickness T2 that is less than the first thickness T1. In certain embodiments, the distal tip 150 has a circular cross-section such that the diameter of the circular cross-section is equal to the thickness of the distal tip. The first thickness T1 can be between about 0.36 mm and about 0.6 mm, such as about 0.45 mm. The second thickness T2 can be 0.3 mm or less, such as 0.25 mm or less, such as 0.2 mm or less. The distal portion 150b has a length L that extends to the distal end of the distal tip 150. The length L can be between 0.25 mm and about 1 mm, such as between about 0.25 mm and about 0.75 mm, such as between about 0.4 mm and about 0.6 mm, such as about 0.5 mm.

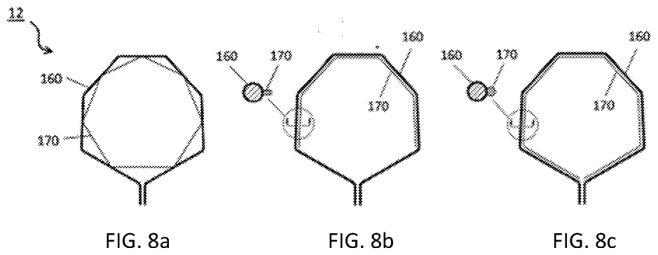

In some embodiments, such as those shown as FIGS. 8a-8c, the loop 12 includes an inner loop 170 that can act as a cutting section and an outer loop 160. The inner loop 170 is bonded (i.e., welded or other known suitable bonding methods) to the outside loop 160. The inner loop 170 comprises a thinner wire that is configured to be more effective for cold cutting. The outer loop 160 is thicker and stiffer than the inner loop 170. The outer loop 160 allows the loop 12 to indent and recruit tissue. Referring to FIG. 8b, in some embodiments, the inner loop 170 only covers the distal/mid portion of the loop 12, where cutting tends to occur. Referring to FIG. 8a, in some embodiments, the inner loop 170 is a full loop, since this may make fixturing/manufacture more efficient. Referring to FIG. 8c, in some embodiments, the inner loop 170 has a round cross-section. Referring to FIG. 8b, in some embodiments, the inner loop 170 is a flat or square wire. It should be understood that the loop 12 can include any combination of the features described with reference to FIGS. 8a-8c.

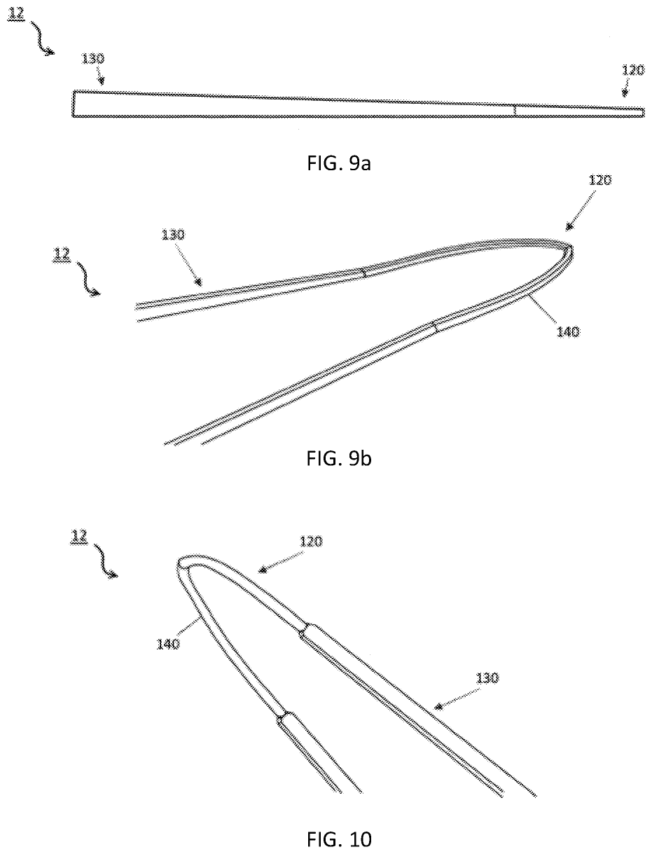

Referring to FIGS. 9a and 9b, the loop 12 may have a tapered or ground wire. The proximal portion 130 is thicker than the distal portion 120. The tool 10 may also include mechanisms for promoting positioning of the loop 12 within the body, such as a rotatable link 22. In some embodiments, the link 22 is of sufficiently less torsional rigidity than the legs of the loop 12.

Referring to FIG. 10, the loop 12 may include a cable or monofilament loop with a coined proximal portion 130 and non-coined distal portion 120.

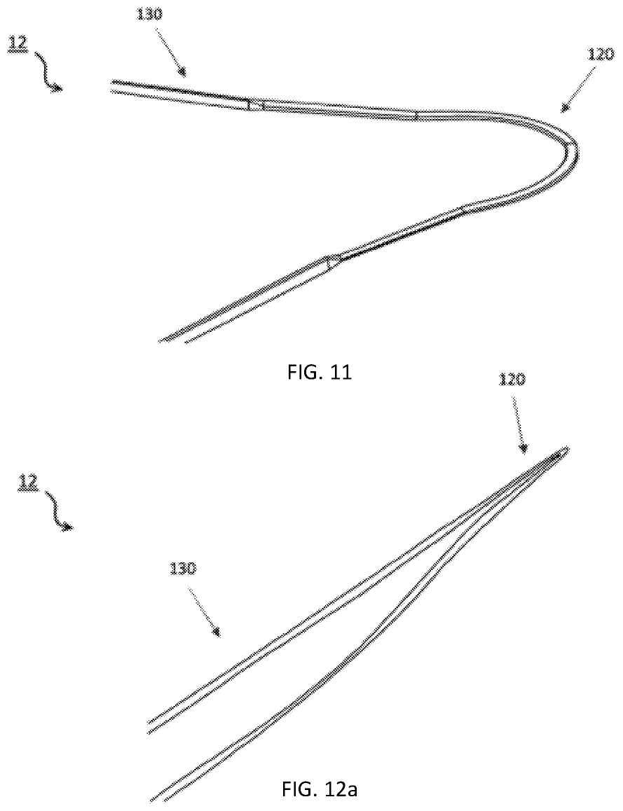

Referring to FIG. 11, the loop 12 may have a cable or monofilament loop with a coined distal portion 120, which creates a sharp inner edge to provide for cutting action. In some embodiments, the proximal portion may also be coined in a perpendicular plane to the distal coined portion.

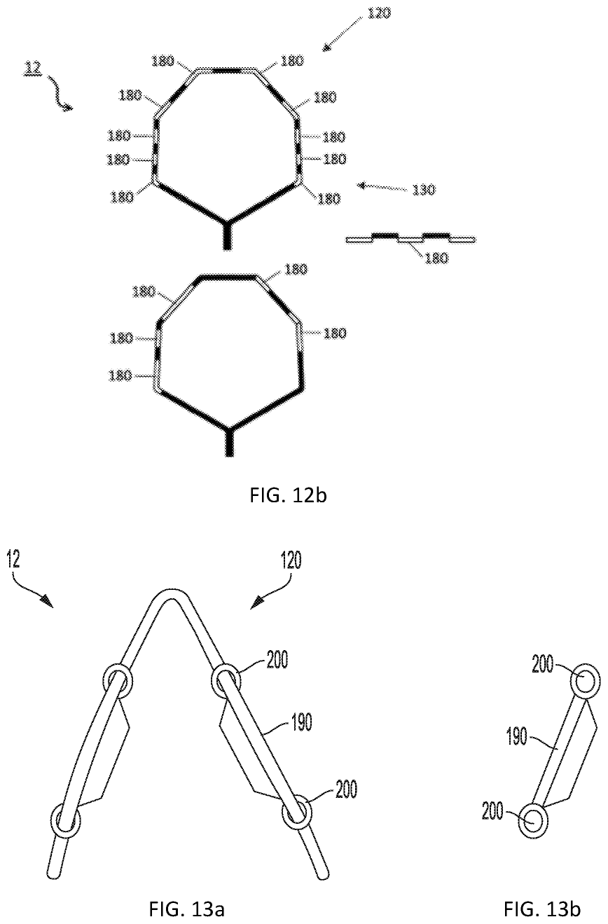

Referring to FIGS. 12a and 12b, the loop 12 can be coined at various locations 180 so as to be advantageous for cutting or grasping tissue. It may be several small level and cross-sectional changes, a few longer sections and may be either symmetrical or asymmetrical with respect to the loop centerlines (vertical and horizontal).

Referring to FIG. 13, the distal portion 120 has one or more cutting elements 190 with eyelets 200. The eyelets 200 could have the wire slid through and then crimped, welded, glued or otherwise bonded in place. The eyelets 200 could be any opening shape, such as round or square.

Referring now to FIG. 4, in the exemplary loop 12, the wire is bent at the distal tip 150 to form a torsion tip, or a nearly 360° circular tip. The tip allows for dissipation of stress through the snare, something that is of particular importance when cold snaring because of the relatively larger forces used compared to hot snaring. One feature to help to facilitate this is to use a multifilament cable. The strands in this type of cable move relative to one another and allow for stresses to be more evenly spread across the snare body. To achieve the same effect in monofilament wire, one can create a loop that mimics a standard torsion spring without increasing the cutting thickness of the tip. Because the tip forms a larger radius than a simple bend, stress is distributed more evenly around the entire circumference upon closing, minimizing plastic deformation.

The distal tip 150 of the tool may have alternative shapes. US 2014/0052142 A1 and US2015/0066045 A1 disclose multiple distal loop or coil tip designs, the contents of which are incorporated herein. The wire may form a torsion coil tip with a 180° bend. In another distal tip, the wire within the 180° bend may be in a landscape orientation. In another exemplary distal tip, the loop is formed by a wire having two portions. The two wire portions form an atraumatic tip at a distal most point. As assembled, the wire portions function as a loop in the same way as discussed herein.

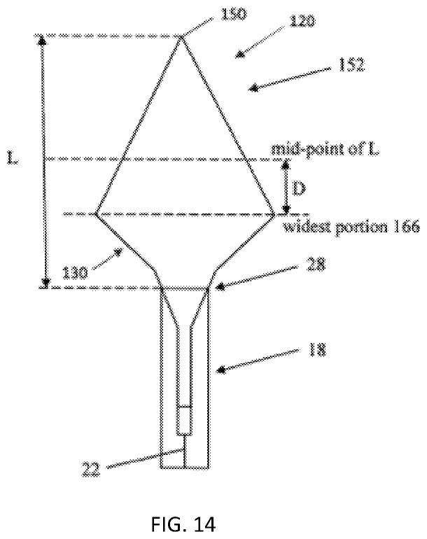

Another aspect of the present subject matter is to provide a new shape of the loop. Referring to FIG. 14, in some embodiments, the loop 152 comprises a widest portion 166 and a length L measured between a proximal end 130 and a distal end 120. The proximal end 130 is defined where the loop begins to close during retraction into the conduit 18, irrespective of where the loop connection physically may occur. The distal end 120 is defined by the most distal end, or ends, of the loop 152. In any instance where there are proximal legs that are excessively long, and/or the distal tip is inverted or of any other unusual geometry, the midpoint shall be considered only with respect to the broad portions of the form, which is defined between the proximal end 130 and the distal end(s) 120. The widest portion 166 of the loop 152 is more proximal to the tubular member 18 than the mid-point of the length L of the loop 152. In other words, the widest portion 166 is closer to the tubular member 18 than the mid-point of the length L of the loop 152, such that the loop 152 is easier to be controlled during the procedures. In some embodiments, the distance D from the mid-point of the length L of the loop 152 to the widest portion 166 of the loop 152 is about 3%-45% of the length L. In some embodiments, the distance D from the mid-point of the length L of the loop 152 to the widest portion 166 of the loop 152 is about 10%-35% of the length L. In some embodiments, the distance D from the mid-point of the length L of the loop 152 to the widest portion 166 of the loop 152 is about 12%-25% of the length L.

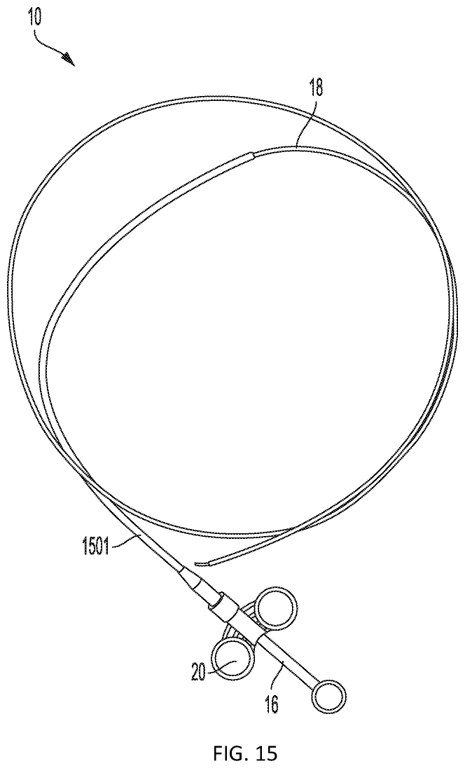

Referring to FIG. 15, another exemplary embodiment of a tissue removal tool 10 includes a body 16, a handle 20, a link (not shown), a conduit 18, a reinforcement member 1501, and a loop (not shown). In certain embodiments, the tissue removal tool 10 is configured to be used with an endoscope. The body 16, handle 20, link, conduit 18, and loop can take any suitable form, such as, for example, any form described in the present application. In various embodiments, a proximal end of the link is connected to the handle 20 and a distal end of the link is attached to the loop such that movement of the handle 20 causes the loop to move between an open position and a closed position by way of the link. The conduit 18 is connected to the body 16 and defines a lumen with an opening at a distal end. The link extends through the conduit 18, and the loop is housed within the conduit 18 when in the closed position and deployed from the conduit 18 when in the open position.

The conduit 18 may be any suitable, small-diameter tube formed of a low-friction flexible material such as, for example, polytetrafluorethylene, high density polyethylene, polyether block amide, polyether ether ketone, coated or uncoated metal, or other comparable materials. In certain embodiments, the conduit 18 may be made of a combination of polytetrafluorethylene and another material, such as, for example, glass, carbon, graphite, molybdenum, wollastonite, polyimide, high density polyethylene, nylon (polyamides), Pebax, PEEK, and other such materials. In some embodiments, the conduit 18 may be made of a polymeric surface with metallic reinforcement, such as, for example, a stainless steel tube, coil, or braid. In alternative embodiments, the metallic reinforcement may be replaced with a high strength non-conductive material, such as liquid crystal polymer.

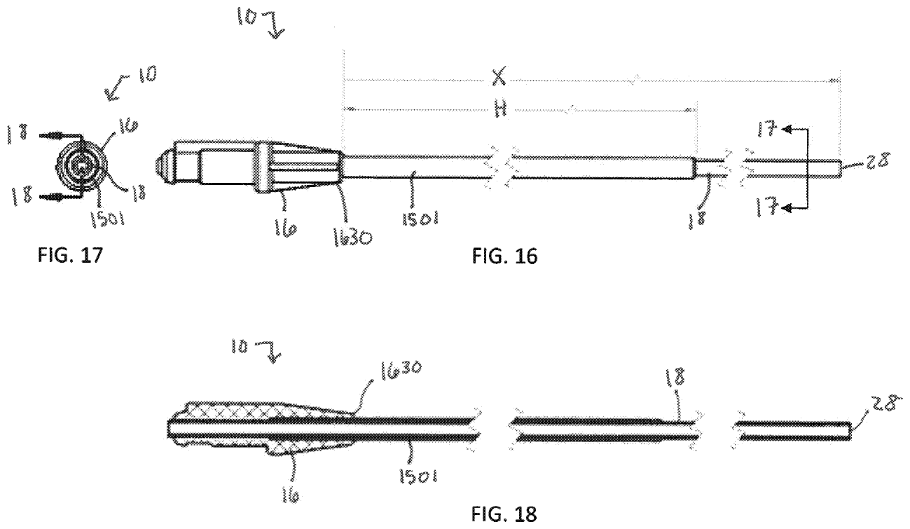

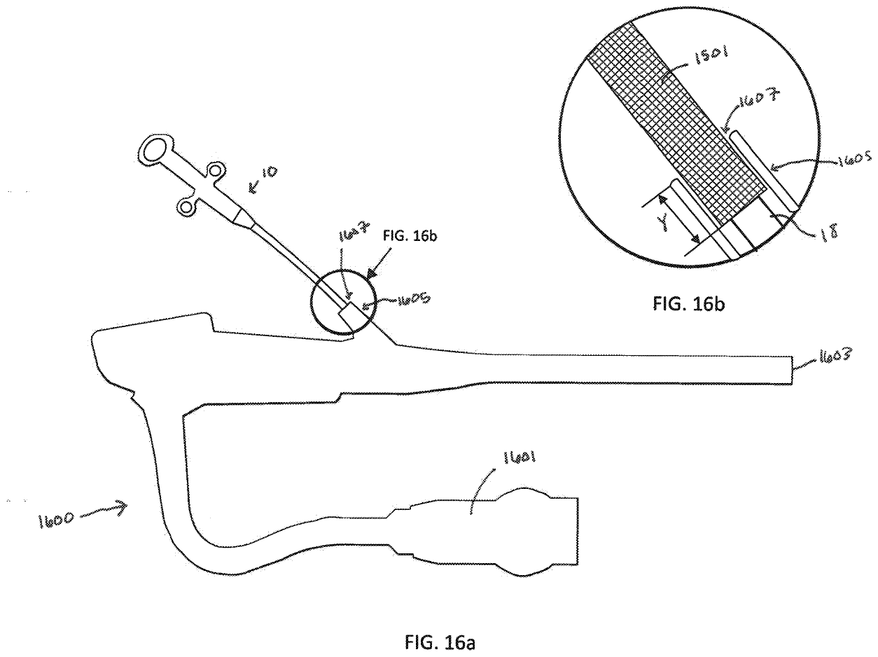

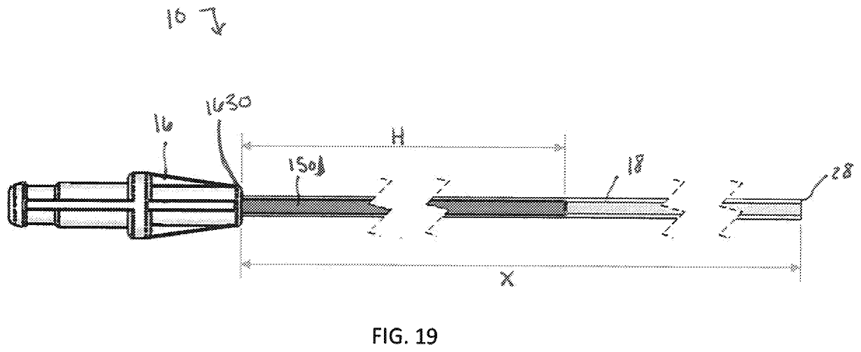

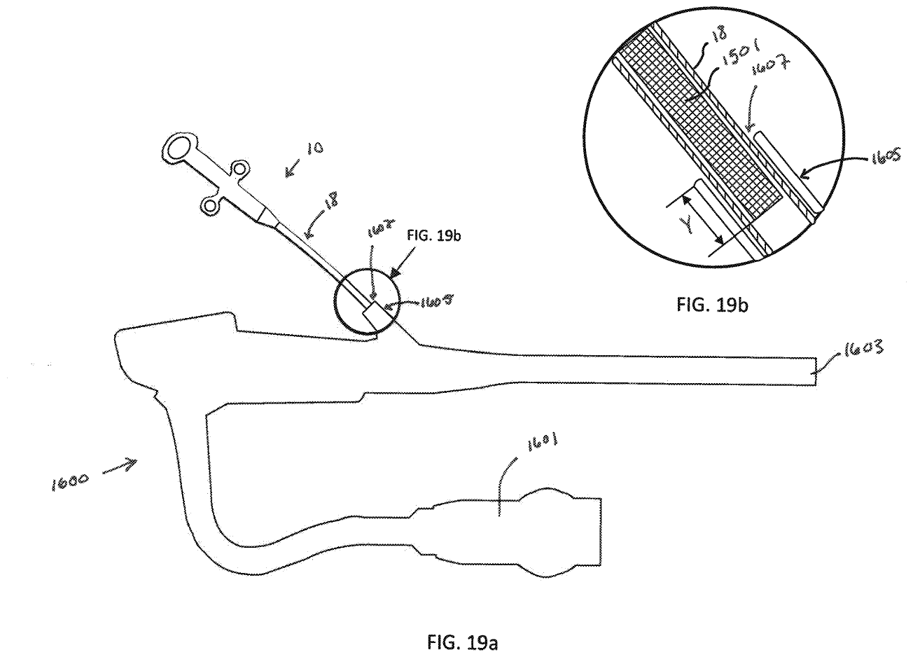

The reinforcement member 1501 is configured to reinforce the conduit 18 to prevent the conduit from buckling or deflecting during use of the tissue removal tool 10. That is, as the tool 10 is being used to collect tissue from a patient, the loop 12 is placed around the tissue to be removed such that the loop can cut the tissue, and this engagement between the tissue and the loop typically allows for axial loading of the loop and, consequently, the link 22 and conduit 18. The reinforcement member 1501 prevents the conduit 18 from buckling or deflecting due to this axial loading. The reinforcement member 1501 may be external to the conduit 18 (as shown in FIGS. 16-18) or may be internal to the conduit (as shown in FIG. 19).

In various embodiments in which the reinforcement member 1501 is internal to the conduit 18, the interaction between the link 22 and the reinforcement member 1501 is dependent on the material and geometry of the link 22 and the reinforcement member 1501. In some embodiments, the link 22 may be coated so as to reduce friction and insulate the link 22 from other components that may be electrically coupled to the link 22. In other embodiments, the reinforcement member 1501 may be non-conductive or otherwise electrically isolated from the link 22. In certain embodiments, the inner diameter of the reinforcement member 1501 is equivalent, or nearly equivalent, to the outer diameter of the link 22, which increases the mechanical contribution from the reinforcement member 1501 by increasing the moment of inertia of the reinforcement member 1501. For example, the difference between the inner diameter of the reinforcement member 1501 and the outer diameter of the link 22 may be less than or equal to 0.035 inches, such as less than or equal to 0.031 inches, such as less than or equal to 0.025 inches, such as less than 0.02 inches, such as less than or equal to 0.015 inches, such as less than or equal to 0.01 inches. The equivalence, or near equivalence, between the inner diameter of the reinforcement member 1501 and the outer diameter of the link 22 allows the full benefit of the reinforcement member 1501 to be realized, as compared to embodiments having a greater difference between the inner diameter of the reinforcement member 1501 and the outer diameter of the link 22. In addition, the minimal clearance available for the link 22 to move inside of the reinforcement member 1501 reduces the lost motion of the link 22 within the tissue removal tool 10, which allows for improved deployment of the loop 12.

In some embodiments, the inner diameter of the conduit 18 is equivalent, or nearly equivalent, to the outer diameter of the reinforcement member 1501, which increases the mechanical contribution from the reinforcement member 1501 by increasing the moment of inertia of the reinforcement member 1501 and removing space for the conduit 18 to deform inwardly. For example, the difference between the inner diameter of the conduit 18 and the outer diameter of the reinforcement member 1501 may be less than or equal to 0.02 inches, such as less than or equal to 0.015 inches, such as less than or equal to 0.01 inches.

Referring to FIGS. 16-18, in certain embodiments, the reinforcement member 1501 is external to the conduit 18. In these embodiments, the reinforcement member 1501 can be a reinforced polymer tubing (e.g., a braid, coil, etc.), a metallic tube or tube like structure (e.g., a braid, coil, etc.), or a polymer tubing (e.g., extrusion, heat shrink, etc.), or any other tubing of sufficient compressional strength that prevents the conduit 18 from buckling, deflecting, bending or shrinking. In embodiments in which the reinforcement member 1501 is made of a metal material, the flexural modulus of the reinforcement member 1501 may be between 50 times greater and 200 times greater than the flexural modulus of the conduit 18. In embodiments in which the reinforcement member is made of a polymer material, the flexural modulus of the reinforcement member 1501 may be between 2 times greater and 10 times greater than the flexural modulus of the conduit 18. In some embodiments, the flexural modulus of the reinforcement member 1501 may be equal to the flexural modulus of the conduit 18.

In various embodiments, the reinforcement member 1501 can be made of a non-conductive material (e.g., a reinforced polymer tubing, a polymer tubing, a polymer with glass or ceramic filler, etc.). A non-conductive reinforcement member is advantageous in embodiments for a hot tissue removal device because the reinforcement member prevents the travel of electricity from the hot tissue removal device to a user (e.g., a doctor or nurse) or patient during use of the device. That is, a conductive reinforcement member that is not electrically isolated from the link 22 and loop 12 can increase the likelihood of a current leakage (as a result of fluid exiting the tissue removal tool 10), which could cause harm to the patient or a device user. Rather than electrically isolating the link 22 and loop 12 from a conductive reinforcement member, which would increase costs as well as the manufacturing complexity of the tissue removal tool 10, it is advantageous to utilize non-conductive materials for the reinforcement member 1501.

The reinforcement member 1501 may be attached to the conduit 18 along the entire length of the reinforcement member 1501, or the reinforcement member 1501 may be attached to the conduit 18 along only a portion of the length of the reinforcement member 1501. The reinforcement member 1501 may connect to the conduit 18 by any suitable means, such as, for example, an adhesive connection, a friction fit connection, a heat bonded connection, an over-molded connection, or any other suitable connection. In embodiments in which the reinforcement member 1501 and the conduit 18 are both made of a polymer material, the reinforcement member 1501 and the conduit may be connected by a heat bonded connection or an over molded connection due to the processability of the polymer material. In alternative embodiments, the reinforcement member 1501 may not be connected to the conduit 18.

Referring to FIGS. 16-18, in some embodiments, the reinforcement member 1501 is disposed around a proximal portion of the conduit 18. For example, the conduit 18 and the reinforcement member can be attached to the distal end 1630 of the body 16. In the illustrated embodiment, the conduit 18 and the reinforcement member 1501 extend into the body 16. The reinforcement member 1501 may connect to the body 16 by any suitable means, such as, for example, an adhesive connection, a friction fit connection, a heat bonded connection, an over-molded connection, or any other suitable connection. The conduit 18 extends a length X from the body 16 to a distal end 28, and the reinforcement member 1501 extends a length H from the body 16. The ratio of the length X to the length H can be, for example, between about 1:1 and about 3:1. While the reinforcement member 1501 is shown as being disposed around the proximal portion of the conduit, it should be understood that the reinforcement member 1501 can be attached to any portion of the conduit 18 between the body 16 and the distal end 28 of the conduit 18.

Referring to FIGS. 16a and 16b, the tissue removal tool 10 may be configured to be inserted into an endoscopic device 1600 such that the tissue removal tool can be used during an endoscopic procedure, such as polypectomy. The endoscopic device 1600 has a connection portion 1601 for connecting to one or more components that are used during the procedure. The components may include, for example, a water source, a suction source, an air pump, a light source, or any other component that is used during an endoscopic procedure. The endoscopic device 1600 also includes a nozzle 1603 and one or more channels (not shown) extending from the connection portion 1601 to the nozzle 1603. The one or more channels are in fluid communication with the components that are connected to the connection portion 1601 such that these components are in fluid communication with the nozzle 1603. The endoscopic device 1600 also includes an inlet conduit 1605 that is in communication with one of the channels such that the conduit 18 of the tissue removal tool can be inserted through the inlet conduit 1605, extended through the channel, and extended out of the nozzle 1603. The inlet conduit 1605 has an inlet opening 1607 for receiving the conduit 18 of the tissue removal tool 10.

Referring to FIG. 16b, in certain embodiments, the lengthH (FIG. 16) of reinforcement member 1501 is configured such that the reinforcement member 1501 extends through the inlet opening 1607 and into the inlet conduit 1605. For example, the reinforcement member 1501 may extend a distance Y from the inlet opening 1607 and into the inlet conduit 1605. The distance Y can be, for example, greater than or equal to 0.25 inches, such as greater than or equal to 0.5 inches. In other embodiments, the distance Y may be less than 0.25 inches. In certain embodiments, the distance Y is between about 0.25 inches and about 3 inches. In some embodiments, the distance Y may be equal to zero and the reinforcement member 1501 may be aligned with the inlet opening 1607. Positioning the reinforcement member 1501 in alignment with the inlet opening 1607 or through the inlet opening 1607 and into the inlet conduit 1605 allows the inlet conduit 1605 to support the portion of the conduit 18 of the tissue removal tool 10 that is not supported by the reinforcement member 1501. That is, the inner walls of the inlet conduit 1605 may provide support to the conduit 18 of the tissue removal tool 10. This configuration is advantageous when the tissue removal tool 10 is used to resect tissue because of the compressive forces resulting from this procedure.

Referring to FIG. 19, in some embodiments, the reinforcement member 1501 is internal to the conduit 18. In these embodiments, the reinforcement member 1501 can be composed of the same material as the conduit 18 or a different material. In some of these embodiments, the internal reinforcement member 1501 has a higher modulus than the conduit 18 while also providing enough flexibility to prevent kinking or undue stiffness to the conduit 18. The internal reinforcement member 1501 can be made of, for example, high density polyethylene, Kocetal, stainless steel spring coils (coated or uncoated), stainless steel hypotubes, laser etched/cut stainless steel hypotubes, polyether ether ketone, or any other suitable material having a relatively high modulus. In certain embodiments, the reinforcement member 1501 may be composed of a combination of the above materials and other suitable materials that are layered over each other to both strengthen the composite structure formed between the conduit 18 and the reinforcement member 1501 and to provide the requisite lubricity and geometry that is required to promote operation of the endoscopic device. In some embodiments, the conduit 18 and the reinforcement material 1501 are integrally formed by the multiple layers of different materials. The layered materials may be formed as part of the conduit 18 or as part of the reinforcement member 1501. In embodiments in which the reinforcement member 1501 is made of a metal material, the flexural modulus of the reinforcement member 1501 may be between 50 times greater and 200 times greater than the flexural modulus of the conduit 18. In embodiments in which the reinforcement member is made of a polymer material, the flexural modulus of the reinforcement member 1501 may be between 2 times greater and 10 times greater than the flexural modulus of the conduit 18. In some embodiments, the flexural modulus of the reinforcement member 1501 may be equal to the flexural modulus of the conduit 18.

In various embodiments, the reinforcement member 1501 can be made of a non-conductive material (e.g., a reinforced polymer tubing, a polymer tubing, a polymer with glass or ceramic filler, etc.). A non-conductive reinforcement member is advantageous in embodiments for a hot tissue removal device because the reinforcement member prevents the travel of electricity from the hot tissue removal device to a user (e.g., a doctor or nurse) or patient during use of the device. A non-conductive reinforcement member also will not dissipate electrical energy as heat through the conduit 18 or handle that can cause burns to a user or a patient.

The reinforcement member 1501 may be attached to the conduit 18 along the entire length of the reinforcement member 1501, or the reinforcement member 1501 may be attached to the conduit 18 along only a portion of the length of the reinforcement member 1501. The reinforcement member 1501 may connect to the conduit 18 by any suitable means, such as, for example, an adhesive connection, a friction fit connection, a heat bonded connection, an over-molded connection, or any other suitable connection. In alternative embodiments, the reinforcement member 1501 may not be connected to the conduit 18.

Referring to FIG. 19, in certain embodiments, the reinforcement member 1501 is disposed in a proximal portion of the conduit 18. The conduit 18 and the reinforcement member can be attached to the distal end 1630 of the body 16 by any suitable means, such as, for example, an adhesive connection, a friction fit connection, a heat bonded connection, an over-molded connection, or any other suitable connection. The conduit 18 extends a length X from the body 16 to a distal end 28, and the reinforcement member 1501 extends a length H from the body 16. The ratio of the length X to the length H can be, for example, between about 1:1 and about 3:1. While the reinforcement member 1501 is shown as being disposed in the proximal portion of the conduit, it should be understood that the reinforcement member 1501 can be disposed in any portion of the conduit 18 between the body 16 and the distal end 28 of the conduit 18.

Referring to FIGS. 19a and 19b, the tissue removal tool 10 may be configured to be inserted into an endoscopic device 1600 such that the tissue removal tool can be used during a polypectomy procedure. The endoscopic device 1600 has a connection portion 1601 for connecting to one or more components that are used during the procedure. The components may include, for example, a water source, a suction source, an air pump, a light source, or any other component that is used during a polypectomy procedure. The endoscopic device 1600 also includes a nozzle 1603 and one or more channels (not shown) extending from the connection portion 1601 to the nozzle 1603. The one or more channels are in fluid communication with the components that are connected to the connection portion 1601 such that these components are in fluid communication with the nozzle 1603. The endoscopic device 1600 also includes an inlet conduit 1605 that is in communication with one of the channels such that the conduit 18 of the tissue removal tool can be inserted through the inlet conduit 1605, extended through the channel, and extended out of the nozzle 1603. The inlet conduit 1605 has an inlet opening 1607 for receiving the conduit 18 of the tissue removal tool 10.

Referring to FIG. 19b, in certain embodiments, the length H (FIG. 19) of reinforcement member 1501 is configured such that the reinforcement member 1501 extends through the inlet opening 1607 and into the inlet conduit 1605. For example, the reinforcement member 1501 may extend a distance Y from the inlet opening 1607 and into the inlet conduit 1605. The distance Y can be, for example, greater than or equal to 0.25 inches, such as greater than or equal to 0.5 inches. In other embodiments, the distance Y may be less than 0.25 inches. In certain embodiments, the distance Y is between about 0.25 inches and about 90 inches. In some embodiments, the distance Y may be equal to zero and the reinforcement member 1501 may be aligned with the inlet opening 1607. Positioning the reinforcement member 1501 in alignment with the inlet opening 1607 or through the inlet opening 1607 and into the inlet conduit 1605 allows the inlet conduit 1605 to support the portion of the conduit 18 of the tissue removal tool 10 that is not supported by the reinforcement member 1501. That is, the inner walls of the inlet conduit 1605 may provide support to the conduit 18 of the tissue removal tool 10. This configuration is advantageous when the tissue removal tool 10 is used to resect tissue because of the compressive forces resulting from this procedure.

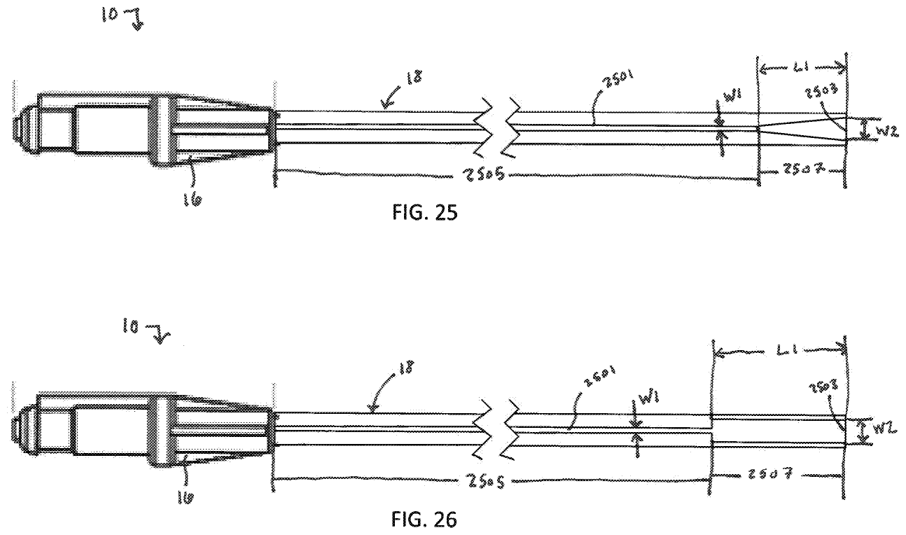

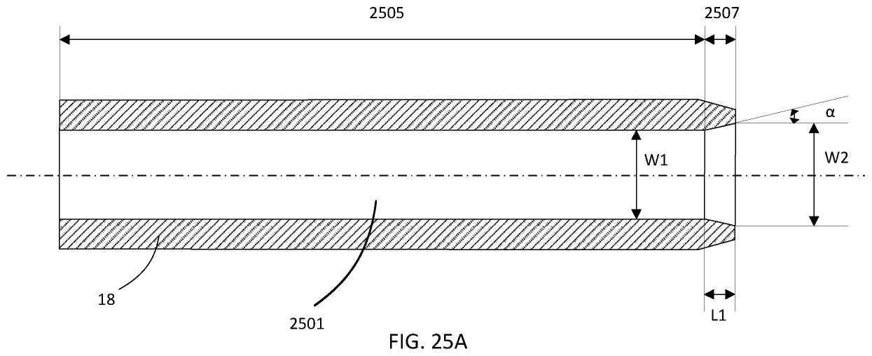

Referring to FIGS. 25 and 26, in certain embodiments, a tissue removal tool 10 includes a body 16, a handle (not shown), a link (not shown), a conduit 18, and a loop (not shown). The tissue removal tool 10 is configured to be used with an endoscope. The body 16, handle, link, and loop can take any suitable form, such as, for example, any form described in the present application. In various embodiments, a proximal end of the link is connected to the handle 20 and a distal end of the link is attached to the loop such that movement of the handle 20 causes the loop to move between an open position and a closed position by way of the link. The conduit 18 may be made of, for example, polytetrafluorethylene, high density polyethylene, polyether block amide, polyether ether ketone, coated or uncoated metal, or other comparable materials. In certain embodiments, the conduit 18 may be made of a combination of polytetrafluorethylene and another material, such as, for example, glass, carbon, graphite, molybdenum, wollastonite, polyimide, high density polyethylene, nylon (polyamides), Kocetal, Pebax, PEEK, and other such materials. In some embodiments, the conduit 18 may be made of a polymeric surface with metallic reinforcement, such as, for example, a stainless steel tube, coil, or braid. In alternative embodiments, the metallic reinforcement may be replaced with a high strength non-conductive material, such as liquid crystal polymer.

The conduit 18 is connected to the body 16 and defines a lumen 2501 with an opening 2503 at a distal end. The lumen 2501 may have a proximal portion 2505 and a distal portion 2507. The proximal portion 2505 has a width W1, and the distal portion 2505 has a maximum width W2 that is greater than the width W1. The width W1 is sized to allow the link 22 to move through the proximal portion 2505 to move the loop between the open and closed positions. The width W1 can be between about 0.05 inches and about 0.06 inches. In certain embodiments, the width W1 is equivalent, or nearly equivalent, to the outer diameter of the link 22. For example, the difference between the width W1 and the outer diameter of the link 22 may be less than or equal to 0.035 inches, such as less than or equal to 0.031 inches, such as less than or equal to 0.025 inches, such as less than or equal to 0.02 inches, such as less than or equal to 0.015 inches, such as less than or equal to 0.01 inches. This small gap between the link 22 and the inner surface of the conduit 18 (defined by the width W1) reduces the amount of lost motion for the link 22 due to slack of the link 22 being disposed within the conduit 18. That is, a large gap between the link 22 and the inner surface of the conduit 18 can allow for slack of the link 22 to snake back and forth within the conduit 18 during deployment, which may prevent the loop 12 from deploying from the conduit 18. Reducing this gap between the link 22 and the inner surface of the conduit 18 prevents reduces the amount of slack of the link 22 within the conduit 18, thus reducing the amount of lost motion. A wall of the conduit 18 that defines the proximal portion 2505 of the inner lumen 2501 may have a thickness (e.g., the thickness T shown in FIG. 25A) that is between about 0.014 inches and about 0.02 inches, such as between about 0.015 inches and about 0.02 inches, such as between about 0.016 inches and about 0.02 inches, such as between about 0.0175 inches and about 0.02 inches, such as about 0.0185 inches. A larger thickness for the conduit 18 prevents the conduit 18 from buckling or deflecting during use of the tissue removal tool 10. That is, a larger thickness for the conduit increases the amount of compressive load that is required to buckle the conduit 18.

Still referring to FIGS. 25 and 26, the distal portion 2507 may have a length L1 that is between about 0.015 inches and about 4 inches, and the distal portion 2507 may have a width W2 that is between about 0.06 inches and about 0.08 inches. In some embodiments, the width W2 and length L1 of the distal portion 2505 is sized to house the loop when the loop is in the closed position. At least a portion of the closed loop may be housed within the distal portion 2507 of the conduit 18, and the loop can be deployed from the distal portion 2507 of the conduit 18 through the opening 2503 to move the loop to the open position.

The conduit 18 may be made from a tube having a lumen 2501 with a uniform width that is equal to the width W1, and the distal end of the tube may be formed mechanically or formed with pressure and heat to create the distal portion 2507. For example, referring to FIG. 25, the distal portion 2507 is created by chamfering the conduit such that the width of the distal portion 2507 gradually increases from the width W1 (at the junction between the proximal portion 2505 and the distal portion 2507) to the width W2 at the opening 2503. Referring to FIG. 26, in other embodiments, the distal portion 2507 is created by reaming the conduit 18 such that the distal portion 2507 has a uniform width that is equal to the width W2. In the embodiments shown in FIGS. 25 and 26, the ratio of the width W2 to the width W1 may be between about 1.1:1 and about 2:1, such as between about 1.25:1 and about 2:1, such as between about 1.4:1 and about 2:1. In some embodiments, the width W2 is between 10 % and 100 % greater than the width W1, such as between about 25 % and about 100 percent, such as between about 40% and about 100%.

Referring to FIG. 25A, in certain embodiments, the conduit 18 is chamfered such that the width of the distal portion 2507 of the inner lumen 2501 gradually increases from the width W1 (at the junction between the proximal portion 2505 and the distal portion 2507) to the width W2 at the opening 2503. The width W1 of the of the proximal portion 2505 of the inner lumen 2501 can be between about 0.05 inches and 0.06 inches, such as between about 0.053 inches and 0.057 inches, such as about 0.055 inches. A wall of the conduit 18 that defines the proximal portion 2505 of the inner lumen 2501 has a thickness T that is between about 0.0175 inches and about 0.02 inches, such as about 0.0185 inches. The width W1 reduces the amount of lost motion of the link 22 within the conduit 18, and the thickness T prevents the conduit 18 from buckling or deflecting during use of the tissue removal tool 10. The width W2 at the opening 2503 of the conduit 18 can be between about 0.06 inches and about 0.075 inches, such as between about 0.065 inches and about 0.072 inches. The length L1 of the distal portion 2507 can be between about 0.015 inches and about 0.06 inches. In certain embodiments, the width of the distal portion 2507 gradually increases by an angle α that is between about 10 degrees and about 20 degrees, such as about 15 degrees.

Referring to 20-21, another exemplary embodiment of a tissue removal tool 10 includes a body (not shown), a handle (not shown), a link 22, a conduit 18, a loop 12, and a connector 2002. In certain embodiments, the tissue removal tool 10 is configured to be used with an endoscope. The body, handle, link 22, conduit 18, and loop 12 can take any suitable form, such as, for example, any form described in the present application. In various embodiments, a proximal end (not shown) of the link 22 is connected to the handle and a distal end 23 of the link 22 is attached to the loop 12 such that movement of the handle causes the loop 12 to move between an open position and a closed position by way of the link 22. The conduit 18 is connected to the body and defines a lumen with an opening 19 at a distal end 28. The link 22 extends through the conduit 18, and the loop 12 is housed within the conduit 18 when in the closed position and disposed outside of the conduit 18 when in the open position. The conduit 18 may be any suitable, small-diameter tube formed of a low-friction flexible material such as, for example, polytetrafluorethylene, high density polyethylene, polyether block amide, or other comparable materials.

The connector 2002 connects the link 22 to the loop 12 such that movement of the link 22 causes the loop 12 to move between the open and closed positions. The connector 2002 includes a first opening 2003 for connecting to the link 22 and a second opening 2005 for connecting to the loop 12. The link 22 can connect to the connector 2002 by any suitable means, such as, for example, an adhesive connection, a threaded connection, a friction fit connection, a soldered connection, a welded connection, or any other suitable connection. The loop 12 can connect to the connector 2002 by any suitable means, such as, for example, an adhesive connection, a threaded connection, a friction fit connection, a crimped connection, a swaged connection, a soldered connection, a welded connection, or any other suitable connection. The connector 2002 can be made of any suitable material, such as, for example, steel, nitinol, or any other suitable material.

Referring to FIG. 20, in certain embodiments, the first opening 2003 is disposed along a first plane 2007, and the second opening 2005 is disposed along a second plane 2009 that is offset from and substantially parallel to the first plane 2007. In this embodiment, the distal end 23 of the link 22 is disposed along the first plane 2007 when the link 22 is connected to the connector 2002, and the loop 12 is disposed along the second plane 2009 when the loop 12 is connected to the connector 2002. Referring to FIG. 21, in some embodiments, a third plane 2111 extends through both the first opening 2003 and the second opening 2005 of the connector 2002 such that the third plane 2111 is substantially perpendicular to the first plane 2007 and the second plane 2009. Orienting the loop 12 so that the third plane 2111 is perpendicular to the second plane 2009 (on which the loop 12 is disposed) allows the loop 12 to have a greater tendency to lay down in a desired position on a patient's tissue during use of the tool 10, which can increase the amount of tissue removed from a patient. That is, the connector maintains the loop in a substantially flat position (rather than an angled position), which allows the loop to engage the base of a polyp and make a substantially straight cut through the polyp, thus removing a larger portion of the polyp.

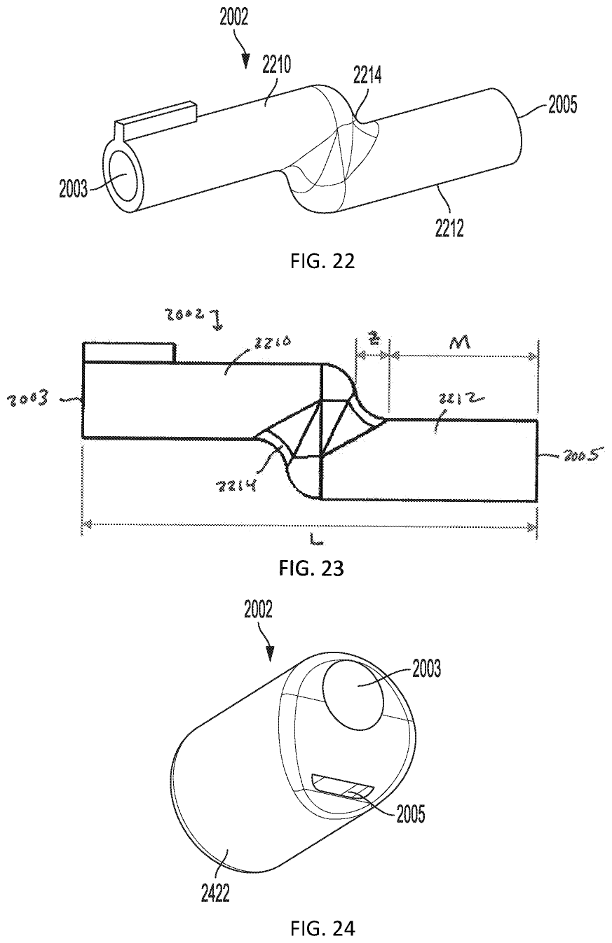

Referring to FIGS. 22 and 23, an exemplary embodiment of a connector 2002 includes a proximal portion 2210 having a first opening 2003 for receiving the link 22 and a distal portion 2212 having a second opening 2005 for receiving the loop 12. In the illustrated embodiment, the connector 2002 includes a center portion 2214 that connects the proximal portion 2210 to the distal portion 2212. In certain embodiments, a length L of the connector 2002 can be between about 1 mm and about 10 mm.

Referring to FIG. 24, another exemplary embodiment of a connector 2002 includes a body 2422, a first opening 2210 that extends through the body 2422, and a second opening 2212 that extends through the body 2422. The body 2422 can be made of any suitable material, such as, for example, steel, nitinol, or any other suitable material. The first opening 2210 is configured for receiving the link 22, and the opening 2212 is configured for receiving the loop 12. The opening 2210 is disposed along a first plane (e.g., plane 2007 shown in FIG. 20), and the opening 2212 is disposed long a second plane (e.g., plane 2009 shown in FIG. 20) that is offset from and substantially parallel to the first plane. A third plane (e.g., plane 2111 shown in FIG. 21) extends through a center of both openings 2210, 2212 such that the third plane is substantially perpendicular to the first and second planes.

It should be understood that some or all of the features described above may be applied to any suitable endoscopic devices or combination of endoscopic devices, such as snare-needle device, a multistage snare, or an endoscopic retrieval device.

A person skilled in the art should understand that although the above-described snare features are designed for cold cutting, they may also be utilized with electrocautery without compromising the features that make them useful for gathering and cutting tissue.

A person skilled in the art should understand that the endoscopic device described in the present subject matter is not necessary to comprise the support assembly (including the base and the elongated tubular member) and/or the transmitting assembly (including the handle and the link). A handle may be formed by or connected to the proximal end of the loop.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts.

While various alternative embodiments as to the various aspects, concepts and features of the inventions (such as alternative materials, structures, configurations, methods, circuits, devices and components, alternatives as to form, fit and function, and so on) may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the general inventive concepts even if such embodiments are not expressly disclosed herein. Additionally, even though some features, concepts or aspects of the inventions may be described herein as being a preferred arrangement or method, such description is not intended to suggest that such feature is required or necessary unless expressly so stated.

Exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

1. A tissue removal tool for use with an endoscope, the tissue removal tool comprising:
a body;
a conduit attached to the body, the conduit defining an inner lumen, the inner lumen having a chamfered distal end, wherein a width of the chamfered distal end gradually increases along a length of the chamfered distal end from a first width to a second width;
a loop that is movable between an open position in which the loop is at least partially disposed outside of the conduit and a closed position in which the loop is disposed within the conduit; and
a transmitting assembly comprising a handle and a link, wherein the handle is connected to the body, wherein the link has a first end attached to the handle and a second end attached to the loop such that movement of the handle causes the loop to move between the open and closed positions.

2. The tissue removal tool according to claim 1, wherein the length of the chamfered distal end is between about 0.015 inches and about 0.0185 inches.

3. The tissue removal tool according to claim 1, wherein the first width is between about 0.05 inches and 0.06 inches.

4. The tissue removal tool according to claim 1, wherein the second width is between about 0.06 inches and 0.075 inches.

5. The tissue removal tool according to claim 1, wherein the width of the chamfered distal end gradually increases by an angle of between about 10 degrees and about 20 degrees.

6. The tissue removal tool according to claim 1, wherein the inner lumen of the conduit has a proximal portion having proximal width.

7. The tissue removal tool according to claim 6, wherein the proximal width is equal to the first width of the chamfered distal end.

8. The tissue removal tool according to claim 6, wherein the proximal width is between about 0.05 inches and 0.06 inches.

9. The tissue removal tool according to claim 6, wherein a wall of the conduit defining the proximal portion of the inner lumen has a thickness of between about 0.0175 inches and about 0.02 inches.

10. The tissue removal tool according to claim 6, wherein a difference between the proximal width of the proximal portion of the inner lumen and an outer diameter of link of the transmitting assembly is less than or equal to 0.035 inches.

11. The tissue removal tool according to claim 1, wherein a ratio of the second width to the first width of the chamfered distal end is between about 1.1:1 and about 2:1.

12. A tissue removal tool for use with an endoscope, the tissue removal tool comprising:
a body;
a conduit attached to the body, the conduit defining an inner lumen, wherein the inner lumen has a proximal portion having a first width and a distal portion having a second width that is greater than the first width;
a loop that is movable between an open position in which the loop is at least partially disposed outside of the conduit and a closed position in which the loop is disposed within the conduit; and
a transmitting assembly comprising a handle and a link, wherein the handle is connected to the body, wherein the link has a first end attached to the handle and a second end attached to the loop such that movement of the handle causes the loop to move between the open and closed positions.

13. The tissue removal tool according to claim 12, wherein a width of the distal portion gradually increases from an initial width to the second width.

14. The tissue removal tool according to claim 13, wherein the ratio of the second width to the initial width is between about 1.1:1 and about 2:1.

15. The tissue removal tool according to claim 12, wherein the second width is between about 0.06 inches and 0.075 inches.

16. The tissue removal tool according to claim 12, wherein the distal portion has a uniform width.

17. The tissue removal tool according to claim 12, wherein the first width is between about 0.05 inches and 0.06 inches.

18. The tissue removal tool according to claim 12, wherein a wall of the conduit defining the proximal portion of the inner lumen has a thickness of between about 0.0175 inches and about 0.02 inches.

19. The tissue removal tool according to claim 12, wherein a difference between the first width of the proximal portion of the inner lumen and an outer diameter of link of the transmitting assembly is less than or equal to 0.035 inches.

20. A tissue removal tool for use with an endoscope, the tissue removal tool comprising:
a body;
a conduit attached to the body, the conduit defining an inner lumen, the inner lumen having a proximal portion having a first width and a chamfered distal end having a width that gradually increases along a length of the chamfered distal end from an initial width to a second width, wherein the first width of the proximal portion is between about 0.05 inches and 0.06 inches, wherein the length of the chamfered distal end is between about 0.015 inches and 0.06 inches, and wherein the second width of the chamfered distal end is between about 0.06 inches and 0.075 inches;
a loop that is movable between an open position in which the loop is at least partially disposed outside of the conduit and a closed position in which the loop is disposed within the conduit; and
a transmitting assembly comprising a handle and a link, wherein the handle is connected to the body, wherein the link has a first end attached to the handle and a second end attached to the loop such that movement of the handle causes the loop to move between the open and closed positions.
